(19) United States
(12) Patent Application Publication
(10) Pub. No.: US 2016/0271145 A1
(43) Pub. Date: Sep. 22, 2016

(54) METHOD FOR ENLARGING THE PARTICLE SIZE OF CRYSTALLINE MICROPARTICLES OF ACTIVE SUBSTANCE

(71) Applicant: Jesalis Pharma GmbH, Jena (DE)

(72) Inventors: Detlef Grawe, Kleinromstedt (DE); Sabine Gliesing, Jena (DE)

(21) Appl. No.: 14/442,354

(22) PCT Filed: Nov. 18, 2013
(86) PCT No.: PCT/EP2013/074044

§ 371 Date: Jun. 10, 2016

(30) Foreign Application Priority Data
Nov. 20, 2012 (DE) 10 2012 221 219.1

(51) Int. Cl. A61K 31/569; A61K 9/00; G01N 15/02; A61K 9/16

(52) U.S. Cl. CPC A61K 31/569; A61K 9/1688; A61K 9/0073; G01N 15/0211

(57) ABSTRACT

A method for enlarging the particle size of crystalline microparticles of active substance by providing

a first suspension prepared from crystalline microparticles of active substance with a first d₅₀ value of 0.5-5 μm, solvent and antisolvent for the active substance, wherein the solubility of the active substance in the solvent-antisolvent mixture of the first suspension is 0.001-0.5 wt. %, such that the average particle diameter (d₅₀ value) at the end or the method is at least 0.03 μm larger than the starting d₅₀ value. The d₅₀ values of a first batch can also serve to control the particle coarsening of a second batch. In addition, particles of fluticasone propionate having an average particle size d₅₀ of 1.0-1.5 μm and simultaneously a narrow particle size distribution with a span <1.35 and high crystallinity with an amorphous fraction of <0.5 wt. % can be prepared.

The invention relates to a method for enlarging the particle size of crystalline microparticles active substance in which a suspension is provided, prepared from crystalline microparticles of active substance with a d₅₀ value of 0.5-5 μm, solvent and antisolvent for the active substance, wherein the solubility of the active substance in the solvent-antisolvent mixture is 0.001-0.5 wt. %, the suspension is mixed, the d₅₀ value of the particles during the process is determined, the suspension is further mixed and then the microparticles of active substance are filtered off under specific conditions discovered in the framework of the invention which do not influence the particle size, redispersed in antisolvent and dried. The d₅₀ value measured during the process serves to control the particle enlargement by adaptation, subject to the measured d₅₀ value, of the mixing time and/or of the solubility of the active substance by addition of solvent and/or antisolvent, such that the average particle diameter (d₅₀ value) at the end of the method is at least 0.03 μm larger than the d₅₀ value at the beginning of the method. The invention further relates to a method for enlarging the particle size of crystalline microparticles of active substance in which d₅₀ values of a first batch serve to control the particle coarsening of a second batch. In addition, the invention relates to particles of fluticasone propionate which can be obtained according to the method and which have an average particle size d₅₀ of 1.0-1.5 μm and simultaneously a narrow particle size distribution with a span ≦1.35 and high crystallinity with an amorphous fraction of ≦0.5 wt. %.

There is a constantly growing need for active substances the particle size and particle size distribution of which are optimally adapted to the pharmaceutical purpose. Grinding methods are often used for this, which, however, lead to unstable and partially amorphized products with a high fraction of fine grains (nanodust) due to their structurally destructive character. Crystallization methods do not have these disadvantages due to their structurally constructive mode of operation. Moreover, they often have the advantage of achieving the desired grain sizes with higher precision and in a narrower distribution than is the case with grinding methods.

However, for certain pharmaceutical applications, e.g. for active substances for pulmonary application, an even more precise and reproducible control of the particle size and particle size distribution by crystallization is often desirable.

Known crystallization methods achieve this particle size range as a rule by setting a very high supersaturation which is as homogeneous as possible, set via intensive mixing with an antisolvent (drowning out), simultaneously applying mechanical energy, as described e.g. in WO 2009/131930, or by subsequent application of energy, as described e.g. in WO 2005/046871. In order to avoid undesired crystal growth and agglomeration, additives (e.g. polymeric cellulose derivatives or surfactants) are used. This can be a disadvantage for certain pharmaceutical applications, as the active substances used must not contain such additives.

A further disadvantage of these methods is above all that the particle formation takes place in a predominantly nuclei formation-oriented manner due to the high level of supersaturation, and thus a relatively wide particle size distribution results and the precision and the reproducibility with which the particle size distribution is generated are limited. Due to the exponential dependency of the nuclei formation rate on the supersaturation, slight variabilities in the process control, in factors related to apparatus, but also in the quality of the raw materials, lead to intolerable fluctuations or batch-to-batch variations of the obtained particle sizes (Hsien-Hsin Tung, et. al, Crystallization of Organic Compounds, Wiley, 2009, p. 103-104). A further problem of these technologies is the scalability. This is risky, above all because of the mixing problems,

In certain pharmaceutical applications, very high demands are made on the reproducibility the particle size and particle size distribution of the active substance. It is thus necessary to keep the pharmaceutical performance of an MDI inhaler (FP—fine particle fraction) within certain permissible limits. This can mean, for example, that the batch-to-batch variation of the average particle size (d₅₀ value) of the active substance may be at most ±0.05 μm in the case of a value close to 1 μm. Known grinding methods and crystallization methods are coming up against their limits here.

The named grinding and crystallization methods have also been applied to fluticasone propionate (fluticasone-17-propionate). Fluticasone propionate is used for pulmonary applications and, for this, should have a d₅₀ value in the range of 1-2 μm, a high crystallinity and a particle size distribution which is as narrow as possible.

A narrow particle size distribution generally has the advantage that the active substance can have a more carefully targeted action, as the release into the body takes place over a shorter time period. In addition, in the case of pulmonary applications, the particles in the boundary ranges of the particle size distribution, i.e. the very small and large particles, do not have the aerodynamic properties of the particles of average size and then do not reach into the fine bronchioles of the lung during application. The kinetics of release and the bioavailability are dependent on the particle size, the particle size distribution and the crystallinity of the particles of active substance.

The standard technology of micronization by means of a jet mill is very problematic especially in the case of the comparatively soft crystals of fluticasone propionate. It is possible to achieve d₅₀ values under 2 μm only with difficulty. In the cases where this is actually managed, the amorphous fractions are particularly large and the width of the particle size distribution displays span values clearly above 2. Due to recrystallization of the amorphous fractions, uncontrolled and undesired coarsenings of the grain size are caused during the pharmaceutical processing and application (Murnane et al., Crystallization and crystallinity of fluticasone propionate, Cryst. Growth & Design, Vol. 8, No. 8, 2008, p. 2753-2764). In order to overcome the disadvantages of micronization, it is suggested in the literature to carry out crystallization methods with the addition of surfactants or polymers as growth inhibitors (Murnane et al., loc. cit.). These lead to an improvement in respect of crystallinity and to average particle sizes below 3 μm. Disadvantages, however, are furthermore the width of the distribution and the lack of precision with which a desired particle size can be set. For instance, no span values smatter than 1.5 are achieved via these methods. In addition, these particles are always contaminated with a certain quantity of surfactant or the growth inhibitor.

Although highly crystalline microparticles of fluticasone proponate with d₅₀ values between 1-2 μm, the span values of which are below those of micronizates, can be achieved using the method of DE 102008037025 A1 , the span values are still 1.7 or above.

A further problem is that fluticasone propionate crystallizes from solvents with needle-like habit (Murnane of al., loc. cit.). Needle-like microparticles, however, have sometimes undesired properties with regard to pharmaceutical processing and application. For instance, the dispersibility in the case of pulmonary application is also dependent on the habit of the particles, In liquid dispersants, e.g. HFA134a, as used in MDIs (metered dose inhaler), particles of fluticasone propionate micronizate and microcrystals of crystallized, needle-like fluticasone propionate tend to form aggregates (Murnane et al., investigations into the Formulation of Metered Dose Inhalers of Salmeterol Xinafoate and Fluticasone Propionate Microcrals Pharmaceutical Research, Vol. 25, No, 10, October 2003, pages 2283-2291). This leads to the impairment of the pharmaceutically effective dose (FP fraction).

The object of the present invention is therefore to overcome the disadvantages of the prior art and to provide a method with which a sought particle size of active substances can be prepared very precisely and which delivers a high crystallinity and narrow particle size distribution of the particles of active substance which can be obtained. A further object of the invention is to provide fluticasone particles which do not have the disadvantages of the prior art.

The object is achieved by a method for enlarging the particle size of crystalline microparticies of active substance, comprising the steps in which
(a) a first suspension is provided, prepared from crystalline microparticles of active substance with a first d₅₀ value of 0.5-5 μm, solvent for the active substance and antisolvent for the active substance, wherein the solubility of the active substance in the solvent-antisolvent mixture of the first suspension is 0.001-0.5 wt. %;
(b) the first suspension is mixed,
(c) the d₅₀ value of the microparticles of active substance contained in the first suspension is determined at least once, wherein a second d₅₀ value is obtained,
(d) the first suspension is further mixed,
(e) the first suspension is filtered off, wherein a filter cake forms, which is washed with antisolvent for the active substance, wherein during the filtering-off a differential pressure of ≦500 mbar is present between the upper side and the lower side of the fitter cake, and the filter cake is not dehydrated to below 80 wt. % antisolvent, relative to the total mass of the filter cake, the obtained filter cake is then suspended in antisolvent, wherein a second suspension is obtained and the d₅₀ value of the microparticles of active substance of the second suspension is determined, wherein a third d₅₀ value is obtained,
   wherein, dependent on the second value, the total mixing time of the first suspension is chosen between 1 and 72 h and/or by addition of solvent and/or antisolvent the solubility of the active substance in the solvent-antisolvent mixture of the first suspension is modified between 0.001 and 0.5 wt. %, such that the third d₅₀ value is at least 0.03 μm larger than the first d₅₀ value, and
(f) the microparticles of active substance of the second suspension are subsequently dried.

The object is further achieved by a method for enlarging the particle size of crystalline microparticles of active substance, comprising the steps in which in a batch A
(a) a first suspension is provided, prepared from crystalline microparticles of active substance with a first value of 0.5-5 μm, solvent for the active substance and antisolvent for the active substance, wherein the solubility of the active substance in the solvent-antisolvent mixture of the first suspension is 0.001-0.5 wt. %,
(b) the first suspension is mixed,
(c) optionally the d₅₀ value of the microparticles of active substance contained in the first suspension is determined, wherein a second d₅₀ value is obtained, the first suspension is further mixed and
(d) the first suspension is then filtered off, wherein a filter cake forms, which is washed with antisolvent for the active substance, wherein during the filtering-off a differential pressure of ≦500 mbar is present between the upper side and the lower side of the filter cake, and the filter cake is not dehydrated to below 80 wt. % antisolvent, relative to the total mass of the filter cake, the obtained filter cake is then suspended in antisolvent, wherein a second suspension is obtained and the value of the microparticles of active substance of the second suspension is determined, wherein a third d₅₀ value is obtained,
   in a batch B
(a) a first suspension is provided, prepared from crystalline microparticles of active substance with a first d₅₀ value of 0.5-5 μm, solvent for the active substance and antisolvent for the active substance, wherein the solubility of the active substance in the solvent-antisolvent mixture of the first suspension is 0.001-0.5 wt. % and wherein in batch B the same active substance, the same solvent and the same antisolvent are used as in batch A,
(b) the first suspension of batch B is mixed,
(c) the first suspension of batch B is then filtered off, wherein a filter cake forms, which is washed with antisolvent for the active substance, wherein during the filtering-off a differential pressure of ≦500 mbar is present between the upper side and the lower side of the filter cake, and the filter cake is not dehydrated to below 80 wt. % antisolvent, relative to the total mass of the filter cake, the obtained filter cake is then suspended in antisolvent, wherein a second suspension is obtained and the d₅₀ value of the microparticles of active substance of the second suspension is determined, wherein a second d₅₀ value is obtained,
   wherein, dependent on the second and/or third d₅₀ value of the microparticles of active substance contained in the first suspension of batch A, the mixing time of the first suspension of batch B is chosen between 1 and 72 h and/or by addition of solvent and/or antisolvent the solubility of the active substance in the solvent-antisolvent mixture of the first suspension in batch B is chosen or modified between 0.001 and 0.5 wt. %, such that the second d₅₀ value of batch B is at least 0.03 μm larger than the first d₅₀ value of batch B,
(d) optionally the second suspension of batch A is added to the second suspension of batch B and
(e) the microparticles of active substance of the second suspension of batch B or of the mixture of the second suspensions of batch A and B are then dried.

The object is also achieved by crystalline particles of fluticasone propionate, the average particle size d₅₀ of which is =1-1.5 μm, the span of which is ≦1.35, wherein the span is defined as (d₅₀-d₁₀)/d₅₀, and the amorphous fraction of which is ≦0.5 wt. %, relative to the total weight of the particles.

With the method according to the invention it is surprisingly possible to achieve a sought particle size very precisely, by starting with particles of active substance the average particle size (d₅₀ value) of which is small, namely lies at least 0.03 μm below a sought d₅₀ value, and by adapting the two process parameters mixing time and solubility of the active substance in the suspension via a measurement of the d₅₀ value carried out during the method. The crystals which can be obtained are additionally highly crystalline and have a very narrow particle size distribution.

By the term “active substance” is meant, in the sense of the invention, a pharmaceutical active substance, i.e. a substance that exerts a physiological action when it is absorbed in sufficient amount by the body of an organism, in particular a mammal, preferably a human.

By microparticles is meant, in the sense of the invention, particles which lie within or below the micrometre range, in particular particles with an average particle size (d₅₀ value) of approximately 0.5-5 μm, “Particles” means a group of particles.

By “crystalline” is meant, in the sense of the invention, particles which are predominantly crystalline. “Amorphous” means, in the sense of the invention, non-crystalline.

The particle size distribution and the diameters of the particles within the framework of the invention are relative to volume. dₓ means that x percent by volume (vol. %) of the particles have a diameter which is smaller than the stated value. With a d₅₀ value of 1 μm therefore 50 vol. % of the particles have a diameter smaller than 1 μm (micrometre). If d₁₀ or d₅₀ is 1 μm, 10 or 90 vol. %, respectively, of the particles have a diameter smaller than 1 μm. The d₅₀ value is also called average particle size or average particle diameter.

The width of the particle size distribution is quantified within the framework of the invention by the span. The span is defined as follows:

span=(d₉₀−d₁₀)/d₅₀.

In the method according to the invention, the d₅₀ growth of the particles in the first suspension is based on the fact that smaller particles in the suspension are more soluble than larger ones. The consequence is that smaller particles dissolve and redeposit onto the larger ones and the particle size grows in this way. The coarser the particles are, the more slowly the process runs. The kinetics of the particle growth are dependent on the solubility ratios, i.e. on the ratio of solvent and antisolvent, and decisively on the type and fractions of by-products which belong to the natural impurity spectrum of the active substance due to the synthesis of the active substance.

It has thus been found, for example, that sulphur-budged dimers of fluticasone propionate and the by-products thereof in an extraordinarily low concentration (0.005-0.05 HPLC area percent relative to the active substance) can change the growth rates of the d₅₀ by almost one order of magnitude, i.e. by a factor of 10. This effect is thus operative far below usual specification thresholds for by-products, which as a rule lie between 0.1% and 1%.

Furthermore, the growth kinetics depend on the antisolvent fraction In the Method according to the invention, the saturation solubility of the active substance is set in the range 0.001 wt. % to 0.5 wt. % via the antisolvent fraction. These relationships can be visualized in a diagram.

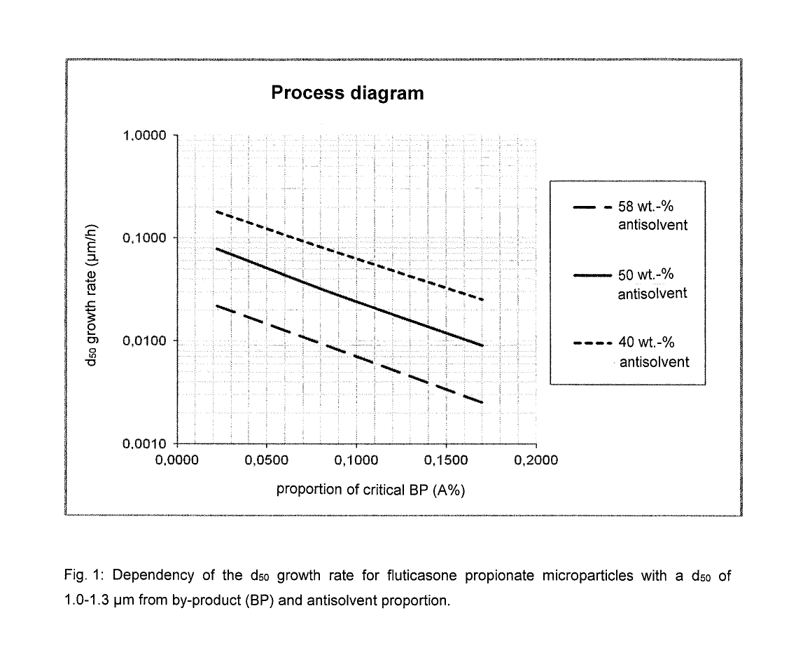

FIG. 1 shows a diagram for the system of fluticasone propionate in acetone as solvent and water as antisolvent, wherein the fraction of critical by-products (BP) is represented in area percent (A %) against the d₅₀ growth rate in μm per hour for three different antisolvent fractions in the suspension.

For a predetermined starting product, i.e. microparticles of a certain active substance with a first d₅₀ value, which has a certain impurity spectrum, a person skilled in the art can establish the required mixing time and/or, as may be necessary, a modification of the solubility in the given limits of 1-72 h and 0.001-0.5 wt. % in a few preliminary tests using the described determination of the d₅₀ value. A modification of the solubility is necessary in particular when the process would otherwise last too long or not long enough. As the by-products, as described above, influence the growth kinetics quite considerably, the process parameters mixing time and solubility must, however, be determined anew for each new batch of a starting product.

By means of the second d₅₀ value, which gives, so to speak, an intermediate prior d₅₀ value to be achieved with the method, the mixing time and solubility of the active substance can be adapted in the already-running process, and thus the third d₅₀ value which is ultimately achieved can be controlled. Furthermore, according to the invention, the second and/or the third d₅₀ value of a first batch (batch A) can be used in order to modify the mixing time and/or solubility in a further batch (batch B). Batch B is separate from batch A. Batch B is preferably started in parallel with or later than batch A. In the latter case, a time advantage is achieved, which is advantageous if the measurement of the second d₅₀ value, inclusive of sample preparation, lasts too long to optimally modify the already-running process. In some cases the third d₅₀ value of batch A is enough to adapt the mixing time and/or solubility in the separate batch B, with the result that the measurement of the second d₅₀ value can be dispensed with. The first, second and third d₅₀ value of batch A and B can also be called first, second and third d₅₀ value (A) and d₅₀ value (B), respectively.

In the method according to the invention, in which two separate batches A and B are used, batch A thus serves primarily for the acquisition of information in order to control the particle size in batch B. For this it is necessary for the same active substance and the same solvent and antisolvent to be present in batch A and B. For an efficient control of the particle enlargement it is preferred that the first d₅₀ value of batch A and that of batch B do not differ by more than 0.1 μm, particularly preferably the first d₅₀ value of batch A and that of batch B are the same. For the same reason, it is preferred that the solubilities of the active substance in the solvent-antisolvent mixture of the first suspension of batch A and of batch B do not differ by more than 10%, particularly preferably the solubilities of the active substance in the solvent-antisolvent mixture of the first suspension of batch A and of batch B are the same.

If the sought particle enlargement is not achieved precisely with one batch, this can be taken into account in further batches. Furthermore, the second suspensions of two or more batches can be mixed in order to achieve the sought particle enlargement, and the mixed second suspensions are then subjected to the drying step. For example, in the method according to the invention the second suspension of batch A can be added to the second suspension of batch B and the thus-obtained second suspension of batch B (mixture) can be dried. In the drying step (e), by the second suspension of batch B is meant the second suspension of batch B with or without admixing of the second suspension of batch A, i.e. the second suspension of batch B or a mixture of the second suspensions of batch B and batch A is dried.

In addition, in batch B between step (b) and (c), the d₅₀ value of the microparticles of active substance contained in the first suspension of batch B can be determined (d₅₀ intermediate value of batch B) and this d₅₀ intermediate value of batch B can then, in addition to the second and/or third d₅₀ value of batch A, be used in order to modify the mixing time and/or the solubility of the active substance of the first suspension of batch B.

The method according to the invention can be carried out discontinuously or continuously. In the case of a continuous procedure, the mixing time is also called the average residence time. A simple agitator vessel with continuous feeds for solvent, antisolvent and first suspension as well as continuous drawing-off of product can be set up for this, for example.

The mixing of the suspension can be effected by stirring, shaking, rotation, ultrasonic treatment, passing gases through, recirculation of the suspension by pumping or other methods of mixing known by a person skilled in the art. Stirring of the suspension is preferred. The mixing preferably takes place in such a way that the particles are kept in motion and a mixing which is as intensive as possible is effected without breaking up the particles. The methods according to the invention are preferably carried out at 0-80° C., in particular 10-40° C.

In the context of the invention it was also found that the working-up of a suspension of microparticles of active substance with d₅₀ values in the range of approximately 0.5-5 μm has a significant influence on the d₅₀ value, because e.g. agglomeration and particle size growth occur during the working-up. As a rule, solvents and antisolvents must be removed from the suspension as quickly and fully as possible in order to avoid a downstream growth of the particles. A distillation step for the purpose of separation as a rule lasts too long and carries the danger of an uncontrolled d₅₀ increase due to precipitations and agglomerations. A filtration process in the case of fine microparticles in the range of from 0.5-5 μm is likewise protracted because of the high filter cake resistance. To speed things up, therefore, a high differential pressure (5-10 bar) is, as a rule, applied to nutsch filters during filtrations. However, this leads to a high compression of the filter cake and the resulting moist product can no longer be redispersed into the primary granulation.

It has been surprisingly found that the suspension with microparticles of active substance and a d₅₀ of 0.5-5 μm can be worked up effectively and without a significant change in the average particle size by filtering off the suspension, wherein a filter cake forms, which is washed with antisolvent for the active substance and, during the filtering-off, a differential pressure of ≦500 mbar is applied between the upper side and the lower side of the filter cake. In addition, the filter cake is not dehydrated to below 80 wt. % antisolvent relative to the total mass of the filter cake, in order to avoid caking of the particles. The filter cake is preferably not dehydrated to below 80 wt. % antisolvent in the whole working-up step, i.e. from the filtering-off to the renewed suspension in antisolvent. The residual moisture is preferably ≧85 wt. % antisolvent relative to the total mass of the filter cake. The obtained filter cake is then suspended (redispersed) in antisolvent and subsequently the microparticles of active substance of this suspension are dried. The filter cake redispersed in antisolvent is eminently suitable for determining the achieved d₅₀ value of the microparticies of active substance (third d₅₀ value). The differential pressure between the upper side and the lower side of the filter cake during the filtration is preferably ≦400 mbar (millibar), particularly preferably 5-200 mbar, in particular 10-50 mbar.

A rotor-stator disperser, e.g. an Ultra Turrax stirrer or a colloid mill is advantageously used for the redispersing. During the redispersing, the weight fraction of the microparticles in the antisolvent is advantageously set to approximately 0.5-10 wt. %, in particular 3-7 wt. % in order to obtain a precise and reproducible measurement result.

The suspending in the method according to the invention is preferably effected by stirring, in particular using a rotor-stator device, e.g. an Ultra Turrax stirrer or a colloid mill. The drying of the suspension and thus the drying of the particles according to step (f) and (e), respectively, can be effected with the usual methods known to a person skilled in the art, for example by spray-drying, evaporation drying, lyophilization or other types of solvent and antisolvent removal.

In order to keep variations due to the sample preparation as small as possible during the measurement of the second d₅₀ value, it is preferred according to the invention that, for the measurement of the second d₅₀ value, microparticles of active substance are removed from the first suspension, filtered off and washed with antisolvent for the active substance, and then the d₅₀ value is determined. Systematic errors due to the method can thereby be avoided. For the same reason, the first d₅₀ value is preferably determined in this way.

By the method according to the invention, the d₅₀ value of the microparticles of active substance used is enlarged by at least 0.03 μm, i.e. the microparticles of active substance are correspondingly coarsened. This means that the third d₅₀ value is at least 0.03 μm larger than the first d₅₀ value. The third d₅₀ value is preferably at least 0.05 μm, in particular at least 0.08 μm, particularly preferably 0.05- 1 μm, in particular 0.1-1 μm larger than the first d₅₀ value. The first d₅₀ value is preferably 0.8-2 μm, in particular 0.9-1.4 μm.

Suitable solvents for the method according to the invention are the liquid solvents known to a person skilled in the art, in particular alcohols, ketones and ethers, for example methanol, ethanol, isopropanol, acetone and diethyl ether. Water is preferably used as antisolvent. By an antisolvent is meant, within the meaning of the invention, a liquid in which the active substance dissolves poorly. The solubility should be less than 0.5 g active substance per litre of antisolvent. Mixtures of several solvents and/or several antisolvents can also be used. Solvents and antisolvents are preferably miscible in the method according to the invention, i.e. they can form a homogeneous mixture.

For an efficient and cost-effective procedure, the solids fraction in the first suspension is preferably in the range of from 0.2-20 wt. %, in particular 0.5-10 wt. %, in each case relative to the total mass of the suspension. Time periods of 1-72 h are suitable as total mixing time for the first suspension, wherein a total mixing time of 5-30 h is preferred for an efficient procedure.

Solubilities of the active substance in the solvent-antisolvent mixture of the first suspension of 0.001-0.5 wt. % are suitable for carrying out the method according to the invention. Solubilites of 0.01-0.2 wt. %, in particular 0.02-0.2 wt. % are preferred. By solubility is meant, within the framework of the invention, the usual solubility known to a person skilled in the art, namely the solubility of a substance in thermodynamic equilibrium at room temperature in the solvent-antisolvent mixture. The solubilities are not relative to volume, i.e., they specify the solubility of the active substance in the total volume of the liquid phase of the suspension. It is clear from the stated solubilities that microparticles of active substance are present throughout the method.

An advantage of the method according to the invention is also the high crystallinity of the obtained particles. This is due to the fact that amorphous, i.e. non-crystalline, fractions and small particles (nanofractions) preferably dissolve, structural imperfections are removed during crystallization onto the coarser particles, and the crystals undergo a very uniform growth at very low supersaturation, without agglomeration. The amorphous fraction of the particles which can be obtained according to the method, relative to the total weight of the particles, is preferably ≦0.5 wt. %, i.e. the crystalline fraction is >99.5 wt. %, particularly preferably ≦0.3 wt. %, i.e. the crystalline fraction (proportion) is preferably >99.7 wt. %.

A further advantage of the method according to the invention is the fact that surface-active compounds and polymers can be dispensed with. The first suspension therefore preferably does not contain any surface-active compounds or surfactants and/or does not contain any polymers. It is further preferred that the first suspension does not contain any other excipients.

The active substance is preferably a steroid, in particular a steroid hormone, preferably a gestagen or an oestrogen, or an antiasthmatic, preferably a glucocorticoid. By asteroid is meant a compound which has the carbon skeleton of perhydrogeriated cyclopenta[a]phenanthrene. The active substance is particularly preferably chosen from the group consisting of drospirenone, desogestrel, dienogest, ethinyl estradiol, fluticasone propionate and budesonide. Fluticasone propionate is the most preferred.

The method according to the invention was particularly advantageously able to be applied to a coarsening of the particle size of crystalline fluticasone propionate, as the particles which can be obtained are eminently suitable for pulmonary applications. In the method according to the invention, in which the active substance is fluticasone propionate, independently of each other, preferably the first value is 0.8-1.2 μm, the total mixing time of the first suspension is 3-25 h, the solvent is acetone and the antisolvent is water and the solubility of the fluticasone propionate in the acetone-water mixture of the first suspension is 0.01-0.1 wt. %. For fluticasone propionate, the third d₅₀ value is preferably 0.05-1 μm, in particular 0.1-0.7 μm larger than the first d₅₀ value. The stated values apply both to the method according to the invention in which the mixing time and solubility of the active substance are adapted from the second d₅₀ value in the already-running process and to the method according to the invention in which the second and/or the third d₅₀ values of batch A are used in order to modify the mixing time and/or solubility in batch B.

The method according to the invention allows highly crystalline particles of fluticasone propionate with a d₅₀ value of 1.5 μm or less and a simultaneously very narrow particle size distribution with a span of 1.35 or less to be produced. The invention therefore also relates to particles of fluticasone propionate with d₅₀ =1-1.5 μm, a span ≦1.35 and an amorphous fraction, relative to the total weight of the particles, of ≦0.5 wt. %. d₅₀ is preferably 1-1.4 μm. The span is preferably ≦1.3, in particular 1.1-1.3. The amorphous fraction is preferably ≦0.3 wt. %, i.e., the crystalline fraction is preferably >99.7 wt. %

In a preferred embodiment, the particles of fluticasone propionate according to the invention have a predominantly spherical shape, whereby the processing properties are improved compared with crystallized, needle-like particles of fluticasone propionate, and they can disperse very well in liquid propellants for MDIs (metered dose inhaler), e.g. HFA134a, and do not tend much towards grain coarsening.

The predominantly spherical shape manifests itself in that in this preferred embodiment the ratio of the largest to the smallest size of the individual particles is on average ≦2. This ratio is determined by measurement on a microscope image of the particles, by determining the ratio of the largest to the smallest size of each individual particle for at least 50 randomly selected particles and then ascertaining the average over the at least 50 particles. Preferably, the ratio of the largest to the smallest size of the individual particles is on average ≦1.6.

The purity of the particles of fluticasone propionate according to the invention is preferably >99 wt. %, relative to the total weight of the particles. The purity is determined by HPLC.

Furthermore, the particles of fluticasone propionate according to the invention preferably do not have any surface-active compounds and/or polymers. The particles of fluticasone propionate according to the invention particularly preferably do not have any excipients. Furthermore, the particles of fluticasone propionate according to the invention are preferably present in the thermodynamically most stable crystal modification.

The invention also relates to a pharmaceutical product containing the particles of fluticasone propionate according to the invention, preferably a pharmaceutical product for pulmonary applications. The invention further relates to particles of fluticasone propionate for use in treatment of diseases which can be treated by pulmonary applications, in particular particles of fluticasone propionate for use in the treatment of bronchial asthma and obstructive lung diseases. The invention further relates to the use of the particles of fluticasone propionate according to the invention as a pharmaceutical product for pulmonary applications, in particular the use of the particles of fluticasone propionate according to the invention for the treatment of bronchial asthma and obstructive lung diseases.

It is understood that the features mentioned above and those yet to be explained below can be used not only in the stated combinations but also in other combinations or alone, without departing from the scope of the present invention.

The following examples illustrate the invention.

Within the framework of the invention, the average particle size (d₅₀ values) and the particle size distribution were measured by means of laser diffraction, which provides a distribution of the particle sizes. A Mastersizer E (Malvern Instruments) was used. 5 mg solid is dispersed for 20 min in 5 ml 0.1% aqueous sodium dodecyl sulphate solution using ultrasound (2 mm sonotrode, sonicator UPS 200, Dr. Hielscher GmbH, 30% amplitude) and placed in the measurement cell of the Mastersizer E, an optical concentration of 18% is set and measurement is carried out (Fraunhofer evaluation). The span was calculated from the d₁₀, d₅₀ and d₉₀ values ((d₉₀ −d₁₀);d₅₀ ).

The amorphous fraction of the crystal particles was determined within the framework of the invention by means of dynamic vapour sorption (DVS). A substance sample (50 to 100 mg) is measured in a DVS device of the type DVS Advantage 1 (Surface Measurement Systems Ltd) under the following conditions: 1. dry nitrogen for 3-5 h. 2. 30% relative acetone moisture content for 4-6 h, 3. dry nitrogen for 3-5 h, 4. 85 to 95% relative acetone moisture content for at least 20 h or until a clear decrease in weight as a consequence of recrystallization is to be observed, 5. dry nitrogen for 4-6 h, 6. 30% relative acetone moisture content for 4-6 h. The fraction of the amorphous phase is proportional to the weight difference between point 2 and point 6. The quantification is effected via a calibration function which is determined from mixtures with known amorphous fractions in a manner known to a person skilled in the art.

### EXAMPLE 1

4280 g of a suspension which contains 3.6 wt. % microparticles of fluticasone propionate is prepared via a crystallization process. The d₅₀ value of the particle size distribution is 0.98 μm. The particle size should be set to 1.28 μm with an accuracy of ±0.04 μm. This suspension contains acetone as solvent and water as antisolvent, in the mass ratio 1:1. The total amount of the impurities relevant for growth was determined via HPLC to be 0.087 A%. By addition of water, the acetone content is reduced to 42% and the solubility fluticasone propionate is thus reduced to 0.032 wt. % (according to FIG. 1 a growth rate of 0.010 μm/h is set). After 4.5 h stirring, the second d₅₀ is measured to be 1.12 μm and further stirring was carried out for 17 h with an unchanged acetone content.

Immediately after the end of the stirring time, the batch is filtered using a nutsch filter with a negative pressure of 100 mbar on the filtrate side and washed with 1.0 l water. The moist filter cake has a mass of approx. 1100 g and is transferred into a 3-l container, topped up to 2160 g with water and redispersed with the Ultra Turrax T50 (|KA) at 12,000 1/min for 10 min. After this, the third d₅₀ value is determined to be 1.28 μm from this second suspension. The span value of the particles is 1.28. The habit is evaluated microscopically as stated above (light microscope image). Habit of the particles: spherical; ratio of the largest to the smallest size of the individual particles on average=1.48. The second suspension is then spray-dried and the particles of fluticasone propionate are obtained.

### EXAMPLE 2

5700 g of a first suspension which contains 3.6 wt. % microparticles of fluticasone propionate is prepared via a crystallization process. The d₅₀ value of the particle size distribution is 0.99 μm. The particle size is to be set to a d₅₀ value of 1.28 μm with an accuracy of ±0.04 μm. This suspension contains acetone as solvent and water as antisolvent, in the mass ratio 1:1. The total amount of the impurities relevant for growth was determined via HPLC to be 0.087 A%. By addition of water, the acetone content is reduced to 42% and the solubility of fluticasone propionate is reduced to 0.032 wt. % (according to FIG. 1 a growth rate of 0.010 μm/h is set).

After 4.0 h stirring, the second d₅₀ value is measured to be 1.12 μm and further stirring is carried out for 13 h with an unchanged acetone content.

Immediately after the end of the stiffing time, the batch is filtered using a nutsch filter with a negative pressure of 100 mbar on the filtrate side and washed with 1.5 l water. The moist filter cake has a mass of approx. 1500 g and is transferred into a 5-l container, topped up to 3000 g with water and redispersed with the Ultra Turrax T50 (IKA) at 12,000 1/min for 10 min. After this, the third d₅₀ value is determined to be 1.24 μm from this second suspension. The span value of the distribution is 1.29.

In a subsequent batch carried out 24 h later, a further 4280 g of a first suspension is prepared which contains 3.6 wt. % microparticles of fluticasone propionate. The d₅₀ value of the particle size distribution is 0.97 μm. The particle size is likewise to be set to 1.28 μm with an accuracy of ±0.04 μm. This suspension contains acetone as solvent and water as antisolvent, in the mass ratio 1:1. The total amount of the impurities relevant for growth was determined via HPLC to be 0.087 A%. By addition of water, the acetone content is reduced to 42% and the solubility of fluticasone propionate is reduced to 0.032 wt. % (according to FIG. 1 a growth rate of 0.010 μm/h is set), After 3.5 h stirring, another, intermediate, d50 value is measured to be 1.09 μm. To control this subsequent batch, the third d50 value of the preceding batch is used. As the third d₅₀ value=1.24 μm from the preceding batch lies at the bottom limit of the target range of 1.28±0.04 μm, the stirring time in comparison with the preceding batch is increased to 18 h with an unchanged acetone content.

Immediately after the end of the stirring time, the batch is filtered using a nutsh filter with a negative pressure of 100 mbar on the filtrate side and washed with 1.0 l water. The moist filter cake has a mass of approx. 1100 g and is transferred into a 3-l container, topped up to 2160 g with water and redispersed with the Ultra Tufrax T50 (IKA) at 12,000 1/min for 10 min. After this, the third d₅₀ value is determined to be 1.30 μm from this second suspension. The span value of the distribution is 124. The habit is evaluated microscopically as stated above. Habit of the particles: spherical; ratio of the largest to the smallest size of the individual particles on average=1.45. A mixture of the second suspensions of the preceding and subsequent batch results in the desired d₅₀ value of 1.28 μm. The span value of the distribution is 1.28. The mixture of the second suspensions is then spray-dried.

Comparison example

Reference test according to DE 102008037025 A1

A 100-ml stirred vessel with blade stirrer is charged with 160 g of Y-stabilized zirconium oxide spheres with a diameter of 0.5 mm plus 30 ml of water. A solution of 0.6 g fluticasone propionate in 10 ml acetone is added to these, with stirring. After stirring for 5 min, a solution of 1.8 g fluticasone in 30 ml acetone and in the same ratio 90 ml water are added dropwise within 10 min, with stirring at 1300 rev/min. Simultaneously, product suspension is drawn off, with approximately constant filling level. Addition and product withdrawal can also take place quasicontinuously in smaller portions without any adverse effect on the result, if the above time frame is maintained. After addition, to complete the yield rinsing is carried out with with approx. 60 ml water. Alternatively, the suspension in the crystallizer can be left as starting suspension for the subsequent batch. Altogether, 180 g of suspension with a solids content of 13 mg per ml is obtained. The suspension is then spray-dried Granulometric analysis and span determination were effected by means of laser diffraction (Sympatec Helps, RODOS dispersing system, dispersing pressure 5 bar).

d₅₀=1.47 μm, span 1.78

1-10. (canceled)

11. A method for enlarging the particle size of crystalline microparticles of active substance, the method comprising:
(a) providing a first suspension, the first suspension prepared from crystalline microparticles of active substance with a first d₅₀ value of 0.5-5 μm, solvent for the active substance, and antisolvent for the active substance, wherein the solubility of the active substance in the solvent-antisolvent mixture of the first suspension is 0.001-0.5 wt. %;
(b) mixing the first suspension;
(c) determining at least one time the d₅₀ value of the microparticles of active substance contained in the first suspension to obtain a second d₅₀ value;
(d) further mixing the first suspension:
(e) filtering off the first suspension to form a filter cake, and washing the filter cake with antisolvent for the active substance, wherein during the filtering-off a differential pressure of 500 mbar is present between the upper side and the lower side of the filter cake, and the filter cake is not dehydrated to below 80 wt. % antisolvent, relative to the total mass of the filter cake, suspending the filter cake in antisolvent forming a second suspension, and determining the d₅₀ value of the microparticles of active substance of the second suspending to obtain a third d₅₀ value;
wherein, dependent on the second d₅₀ value, the total mixing time of the first suspension is chosen between about 1 hour and 72 hours and/or by addition of solvent and/or antisolvent the solubility of the active substance in the solvent-antisolvent mixture of the first suspension is modified between 0.001 and 0.5 wt. %, such that the third d₅₀ value is at least 0.03 μm larger than the first d₅₀ value; and
(f) drying the microparticles of active substance of the second suspension.

12. The method of claim 11, wherein the total mixing time of the first suspension is between 5 hours and 30 hours.

13. The method of claim 11, wherein the solubility of the active substance in the solvent-antisolvent mixture of the first suspension is 0.01-0.2 wt. %.

11. The method of claim 11, wherein the first suspension does not contain any surface-active compounds.

15. The method of claim 11, wherein the first suspension does not contain any polymers.

16. The method of claim 11, wherein the active substance is a steroid hormone or a glucocorticoid.

17. The method of claim 11, wherein the active substance is selected from the group consisting of drospirenone, desogestrel, dienogest, ethinylestradiol, fluticasone propionate, and budesonide.

18. The method of claim 11, wherein the active substance is fluticasone propionate.

19. The method of claim 18, wherein the first d₅₀ value is 0.8-1.2 μm, the total mixing time of the first suspension is 3 hours to 25 hours, the solvent is acetone and the antisolvent is water, and the solubility of the fluticasone propionate in the acetone-water mixture of the first suspension is 0.01-0.1 wt. %.

20. A method for enlarging the particle size of crystalline microparticles of active substance, the method comprising:
preparing a batch A, preparation of the batch A comprising:
(a) providing a first suspension, the first suspension prepared from crystalline microparticles of active substance with a first d₅₀ value of 0.5-5 μm, solvent for the active substance, and antisolvent for the active substance, wherein the solubility of the active substance in the solvent-antisolvent mixture of the first suspension is 0.001-0.5 wt. %;
(b) mixing the first suspension;
(c) optionally determining the d₅₀ value of the microparticles of active substance contained in the first suspension to obtain a second d₅₀ value, wherein the first suspension is further mixed after the second d₅₀ value is determined; and
(d) filtering off the first suspension to form a filter cake, washing the filter cake with antisolvent for the active substance, wherein during the filtering-off a differential pressure of ≦500 mbar is present between the upper side and the lower side of the filter cake, and the filter cake is not dehydrated to below 80 wt. % antisolvent, relative to the total mass of the filter cake, suspending the filter cake in antisolvent forming, a second suspension, and determining the d₅₀ value of the microparticles of active substance of the second to obtain a third d₅₀ value; and
preparing a batch B, preparing of the batch B comprising:
(a) providing a first suspension, the first suspension prepared from crystalline microparticles of active substance with a first d₅₀ value of 0.5-5 μm, solvent for the active substance and antisolvent for the active substance forming a solvent-antisolvent mixture, wherein the solubility of the active substance in the solvent-antisolvent mixture of the first suspension is 0.001-0.5 wt. %, and wherein in the batch B the same active substance, the same solvent and the same antisolvent are used as in the batch A;
(b) mixing the first suspension;
(c) filtering off the first suspension forming a filter cake, washing the filter cake with antisolvent for the active substance. wherein during the filtering-off a differential pressure of ≦500 mbar is present between the upper side and the lower side of the filter cake, and the filter cake is not dehydrated to below 80 wt. % antisolvent, relative to the total mass of the filter cake, suspending the obtained filter cake in antisolvent, wherein a second suspension is obtained, and determining the d₅₀ value of the microparticles of active substance of the second suspension to obtain a second d₅₀ value;
wherein, dependent on the second and/or third d₅₀ value of the microparticles of active substance contained in the first suspension of the batch A, the total mixing time of the first suspension of the batch B is chosen between 1 hour and 72 hours and/or by addition of solvent and/or antisolvent the solubility of the substance in the solvent-antisolvet mixture of the first suspension in the batch B is chosen or modified between 0.001 and 0.5 wt. %, such that the second d₅₀ value of the batch B is at least 0.03 μm larger than the first d₅₀ value of the batch B;
(d) optionally adding the second suspension of the match A to the second suspension of the hatch B; and
(e) drying the microparticles of active substance of the second suspension of the batch B.

20. The method of claim 20, wherein the total mixing time of the first suspension is between 5 hours and 30 hours.

22. The method of claim 20, wherein the solubility of the active substance in the solvent-antisolvent mixture of the first suspension is 0.01-0.2 wt. %.

20. The method of claim 20, wherein the first suspension does not contain any surface-active compounds.

24. The method of claim 20, wherein the first suspension does not contain any polymers.

25. The method of claim 20, wherein the active substance is a steroid hormone or a glucocorticoid.

26. The method of claim 25, wherein the active substance is selected from the group consisting of drospirenone, desogestrel, dienogest, ethihylestradiol, fluticasone propionate, and budesonide.

20. The method of claim 20, wherein the active substance is fluticasone propionate.

21. The method of claim 27, wherein the first d₅₀ value is 0.8-1.2 μm, the total mixing time of the first suspension is 3 hours to 25 hours, the solvent is acetone and the antisolvent is water, and the solubility of the fluticasone propionate in the acetone-water mixture of the first suspension is 0.01-0.1 wt. %.

29. A plurality of particles comprising crystalline particles of fluticasone propionate, wherein the average particle size d₅₀ is 1-1.5 μm, the span is ≦1.35, wherein the span is defined as (d₉₀-d₁₀)/(d₅₀, and the amorphous fraction is ≦0.5 wt. %, relative to the total weight of the particles.

30. The plurality of particles of claim 29, wherein the ratio of the largest to the smallest size of the individual particles is on average ≦2.

31. The plurality of particles of claim 29, wherein the plurality of particles are provided in a pharmaceutical composition for pulmonary applications.
